(19) United States
(12) Patent Application Publication
(10) Pub. No.: US 2026/0215947 A1
(43) Pub. Date: Jul. 30, 2026

(54) DELIVERY AID FOR USE WITH A STENT DELIVERY SYSTEM

(71) Applicant: BOSTON SCIENTIFIC SCIMED, INC., Maple Grove, MN (US)

(72) Inventors: Gerard Duignan, Loughrea (IE); Louis McNern, Kinvara (IE); Gary Gilmartin, Foxford (IE)

(73) Assignee: BOSTON SCIENTIFIC SCIMED, INC., Maple Grove, MN (US)

(21) Appl. No.: 19/464,239

(22) Filed: Jan. 29, 2026

Related U.S. Application Data
(60) Provisional application No. 63/751,645, filed on Jan. 30, 2025.

(51) Int. Cl. A61F 2/966

(52) U.S. Cl. CPC A61F 2/966

(57) ABSTRACT

A delivery aid for use with a stent delivery catheter includes a handle, an elongate shaft, a first delivery system receiver attached to the handle, a second delivery system receiver attached to the shaft, and an actuation mechanism configured to translate portions of the catheter relative to each other with the catheter disposed outside of the handle. The delivery aid includes a first receptacle configured to receive a first handle of the catheter, and a second receptacle configured to receive a second handle of the catheter. The actuation mechanism is configured to translate the receptacles relative to each other to deploy a stent from the catheter. A stent delivery system includes the catheter, a stent radially constrained between tubular members of the catheter, and the delivery aid. Actuation of the actuation mechanism is configured to translate the tubular members relative to each other to deploy the stent.

### CROSS REFERENCE TO RELATED APPLICATIONS

The present application claims the benefit of US Provisional Patent Application Serial No. 63/751,645, filed January 30, 2025, the disclosure of which is incorporated herein by reference.

### TECHNICAL FIELD

The present disclosure pertains to medical devices and uses thereof. More particularly, the present disclosure pertains to a delivery aid for use with a stent delivery system for implanting a self-expanding endoprosthesis or stent in a body lumen, and associated methods.

### BACKGROUND

An endoprosthesis may be used in the treatment of body lumens. One type of endoprosthesis used in the repair and/or treatment of diseases in various body lumens is a stent. A stent is a generally longitudinal tubular device formed of biocompatible material which is useful to open and support various lumens in the body. For example, stents may be used in the vascular system, urogenital tract, gastrointestinal tract, esophageal tract, tracheal/bronchial tubes, and bile duct, as well as in a variety of other applications in the body.

In some instances, smooth and accurate deployment of self-expanding endoprostheses or stents may be difficult due to various reasons. In some instances, multiple personnel may be required to operate and control all of the equipment used for delivery of the endoprosthesis or stent in an endoscopic procedure. In some instances, it may be desirable to limit foreshortening. In some instances, it may be desirable to simplify the delivery system. In some instances, it may be desirable to improve deployment accuracy. In some instances, it may be desirable to provide control of stent deployment and placement to the physician.

Of the known medical devices and methods, each has certain advantages and disadvantages. There is an ongoing need to provide alternative medical devices as well as alternative methods for manufacturing and using medical devices.

### SUMMARY

In one example, a delivery aid for use with a stent delivery catheter may comprise a handle, an elongate shaft extending distally from the handle, a first delivery system receiver attached to the handle and configured to releasably couple to a first portion of the stent delivery catheter, a second delivery system receiver attached to the elongate shaft distal of the handle and configured to releasably couple to a second portion of the stent delivery catheter, and an actuation mechanism disposed at least partially within the handle. The actuation mechanism may be configured to axially translate the second portion of the stent delivery catheter relative to the first portion of the stent delivery catheter with the stent delivery catheter disposed entirely outside of the handle.

In addition, or alternatively, to any example disclosed herein, the first delivery system receiver is provided on an exterior surface of the handle.

In addition, or alternatively, to any example disclosed herein, the second delivery system receiver is provided on an exterior surface of the elongate shaft.

In addition, or alternatively, to any example disclosed herein, the first delivery system receiver comprises a first lateral opening configured to receive the first portion of the stent delivery catheter laterally therein.

In addition, or alternatively, to any example disclosed herein, the first portion of the stent delivery catheter is a first handle of the stent delivery catheter attached to a proximal end of an inner tubular member of the stent delivery catheter, wherein the first delivery system receiver includes a first receptacle configured to removably secure the first handle of the stent delivery catheter therein.

In addition, or alternatively, to any example disclosed herein, the second delivery system receiver comprises a second lateral opening configured to receive the second portion of the stent delivery catheter laterally therein.

In addition, or alternatively, to any example disclosed herein, the second portion of the stent delivery catheter is a second handle of the stent delivery catheter attached to a proximal end of an outer tubular member of the stent delivery catheter, wherein the second delivery system receiver includes a second receptacle configured to removably secure the second handle of the stent delivery catheter therein.

In addition, or alternatively, to any example disclosed herein, the actuation mechanism includes a thumb wheel operatively engaged with the elongate shaft.

In addition, or alternatively, to any example disclosed herein, rotation of the thumb wheel is configured to axially translate the second delivery system receiver relative to the first delivery system receiver.

In addition, or alternatively, to any example disclosed herein, the elongate shaft comprises a linear gear and the thumb wheel comprises a circular gear engaged with the linear gear.

In addition, or alternatively, to any example disclosed herein, the handle comprises a visual aid configured to show how much of a stent disposed within the stent delivery catheter has been deployed.

In addition, or alternatively, to any example disclosed herein, the visual aid comprises a window formed in a wall of the handle and a linear scale disposed alongside the window.

In addition, or alternatively, to any example disclosed herein, the first delivery system receiver is detachable from the handle and the second delivery system receiver is detachable from the elongate shaft.

In addition, or alternatively, to any example disclosed herein, and in a second example, a delivery aid for use with a stent delivery catheter may comprise a handle, an actuation mechanism disposed at least partially within the handle, a first receptacle configured to removably receive a first handle of the stent delivery catheter therein, and a second receptacle configured to removably receive a second handle of the stent delivery catheter therein. The actuation mechanism may be configured to axially translate the second receptacle relative to the first receptacle to deploy a stent from the stent delivery catheter.

In addition, or alternatively, to any example disclosed herein, the first receptacle comprises a first lateral opening configured to receive the first handle of the stent delivery catheter laterally therein.

In addition, or alternatively, to any example disclosed herein, the second receptacle comprises a second lateral opening configured to receive the second handle of the stent delivery catheter laterally therein.

In addition, or alternatively, to any example disclosed herein, and in a third example, a stent delivery system may comprise a stent delivery catheter comprising an outer tubular member, an inner tubular member slidably disposed within the outer tubular member, a first handle secured to a proximal end of the inner tubular member, and a second handle secured to a proximal end of the outer tubular member; a stent radially constrained between the inner tubular member and the outer tubular member; and a delivery aid comprising a handle, an actuation mechanism disposed at least partially within the handle, a first receptacle configured to removably receive the first handle of the stent delivery catheter therein, and a second receptacle configured to removably receive the second handle of the stent delivery catheter therein. Actuation of the actuation mechanism may be configured to axially translate the outer tubular member relative to the inner tubular member to deploy the stent.

In addition, or alternatively, to any example disclosed herein, the first receptacle and the second receptacle are configured to position the stent delivery catheter entirely outside of the handle of the delivery aid.

In addition, or alternatively, to any example disclosed herein, the actuation mechanism is configured for measured deployment of the stent from the stent delivery catheter.

In addition, or alternatively, to any example disclosed herein, the handle is configured for ambidextrous operation of the actuation mechanism.

The above summary of some embodiments, aspects, and/or examples is not intended to describe each disclosed embodiment or every implementation of the present disclosure. The figures and detailed description which follow more particularly exemplify these embodiments .

### BRIEF DESCRIPTION OF THE DRAWINGS

The disclosure may be more completely understood in consideration of the following detailed description in connection with the accompanying drawings, in which:

FIGS. 1-2 schematically illustrate selected aspects of a stent delivery system comprising a stent delivery catheter and a delivery aid;

FIG. 3 is a partially exploded view schematically illustrating selected aspects of the delivery aid;

FIG. 4 is a side view schematically illustrating selected aspects of the delivery aid;

FIGS. 5-6 are top views schematically illustrating selected aspects of the delivery aid; FIG. 7 is a top view schematically illustrating selected aspects of the delivery aid; and

FIG. 8 is a side view schematically illustrating selected aspects of an alternative configuration of the delivery aid.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently – such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. It is noted that some reference numbers may be discussed but are not expressly shown with respect to a particular figure. Reference numbers discussed but not expressly shown may be shown in other figures. Similarly, some reference numbers shown but not expressly discussed may be discussed with respect to other figures herein. The systems, devices, and/or methods disclosed herein may provide a number of desirable features and benefits as described in more detail below.

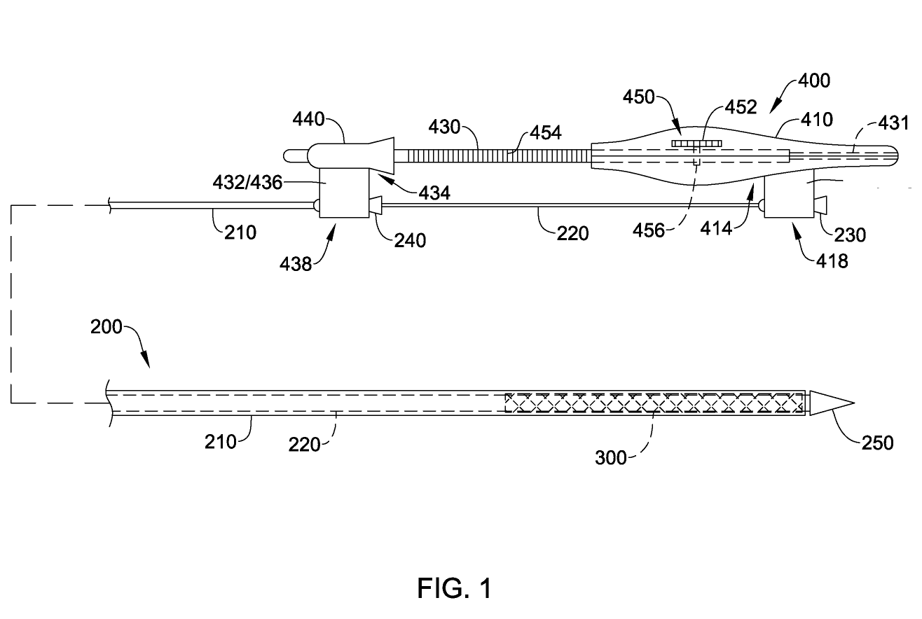

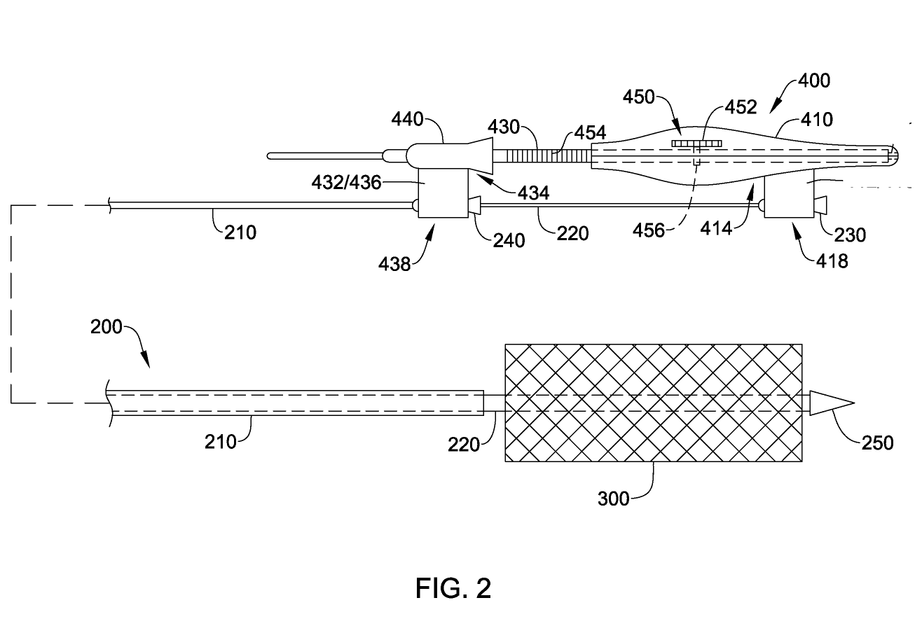

FIGS. 1-2 illustrate selected aspects of a stent delivery system 100. The stent delivery system 100 may comprise a stent delivery catheter 200 configured to deliver and/or deploy a stent 300 at a treatment site. In some embodiments, the stent delivery catheter 200 may comprise an outer tubular member 210 and an inner tubular member 220 slidable disposed within the outer tubular member 210. In some embodiments, the stent delivery catheter 200 may comprise a first handle 230 secured and/or fixedly attached to a proximal end of the inner tubular member 220. In some embodiments, the stent delivery catheter 200 may comprise a second handle 240 secured and/or fixedly attached to a proximal end of the outer tubular member 210. Some suitable but non-limiting examples of materials for the stent delivery catheter 200, the outer tubular member 210, the inner tubular member 220, the first handle 230, and/or the second handle 240 are discussed below.

The stent 300 may be disposable and/or disposed within an annular space between the inner tubular member 220 and the outer tubular member 210 (e.g., within a lumen of the outer tubular member 210 and around the inner tubular member 220) in a radially collapsed configuration and/or a delivery configuration, as seen in FIG. 1. The stent 300 may be radially constrained between the inner tubular member 220 and the outer tubular member 210. The inner tubular member 220 and the outer tubular member 210 may be axially movable relative to each other to load the stent 300 into, and deploy the stent 300 from, the stent delivery catheter 200. In some embodiments, the inner tubular member 220 may comprise a guidewire lumen extending therein. In some embodiments, the stent delivery catheter 200 and/or the inner tubular member 220 may comprise a distal tip 250 disposed at a distal end of the inner tubular member 220. In at least some embodiments, the distal tip 250 may be tapered radially inward in a distal direction and/or may be atraumatic.

In at least some embodiments, the stent 300 may be configured to self-expand from the radially collapsed configuration and/or the delivery configuration (e.g., FIG. 1) toward and/or to a radially expanded and/or a deployed configuration, as seen in FIG. 2, when unconstrained by the outer tubular member 210. Other stent configurations are also contemplated. In some embodiments, the stent 300 may comprise a body portion extending axially from a first end to a second end along a central longitudinal axis. In some embodiments, the first end and the second end may define a length of the stent 300 and/or the body portion. In at least some embodiments, the body portion may be tubular. The body portion may define a lumen extending therethrough from the first end to the second end. In some embodiments, the stent 300 may be self-biased toward and/or to the radially expanded configuration and/or the deployed configuration.

In some embodiments, the stent 300 may comprise a flared first end portion (not shown) and/or a flared second end portion (not shown), wherein an outer diameter of the flared first end portion is greater than an outer diameter of the body portion and/or an outer diameter of the flared second end portion is greater than the outer diameter of the body portion. Other configurations are also contemplated. The flared first end portion may be disposed proximate and/or adjacent the first end. In some embodiments, the flared first end portion may extend from the first end to the body portion. The flared second end portion may be disposed proximate and/or adjacent the second end. In some embodiments, the flared second end portion may extend from the second end to the body portion.

In some embodiments, the outer diameter of the body portion may be generally uniform and/or generally constant along the length of the stent 300 and/or the body portion. In some embodiments, the outer diameter of the body portion may be generally uniform and/or generally constant along the length of the body portion except for the flared first end portion and/or the flared second end portion, where present. In some embodiments, the outer diameter of the body portion may be generally uniform and/or generally constant along the length of the body portion from the flared first end portion to the flared second end portion, where present. Other configurations are also contemplated.

Some suitable but non-limiting examples of materials for the stent 300, such as metallic materials, composite materials, shape memory materials, combinations thereof, etc., are discussed below. In one non-limiting example, the stent 300 may be formed from nickel-titanium alloy (e.g., nitinol). In another non-limiting example, the stent 300 may be formed from nickel-titanium alloy with a platinum core. Other configurations and/or materials are also contemplated.

In some embodiments, the stent 300 may comprise a polymeric covering. In some embodiments, the polymeric covering may be fixedly attached to the stent 300. In some embodiments, the polymeric covering may extend along an inner surface of the stent 300. In some embodiments, the polymeric covering may extend along an outer surface of the stent 300. In at least some embodiments, the stent 300 may be embedded within the polymeric covering. Other configurations, including combinations thereof, are also contemplated. Some suitable but non-limiting examples of polymeric materials for the polymeric covering are discussed below.

The stent delivery system 100 and/or the stent delivery catheter 200 may be configured to deliver the stent 300 to, and/or may be configured to deploy the stent 300 within, a body lumen. The body lumen may be any one of a variety of body lumens, such as but not limited to a blood vessel, esophageal tract, digestive tract, biliary tract, renal tract, etc., as is known in the art. Other configurations are also contemplated.

In some embodiments, the stent delivery catheter 200 may be a standalone stent delivery device configured to be advanced to the body lumen on its own (e.g., without the aid of guide sheath, access sheath, etc.). In some embodiments, the stent delivery catheter 200 may be configured to be advanced to the body lumen within and/or through a tubular member (such as a guide sheath, an access sheath, etc.), a catheter, an endoscope, etc. Other configurations are also contemplated.

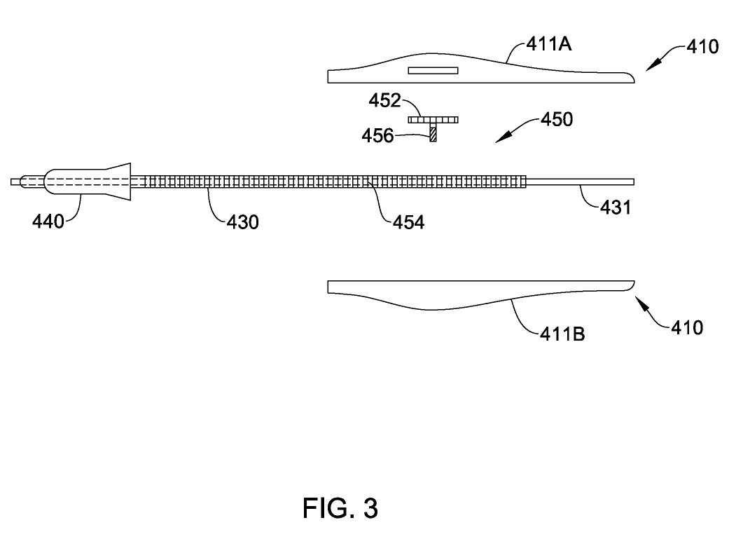

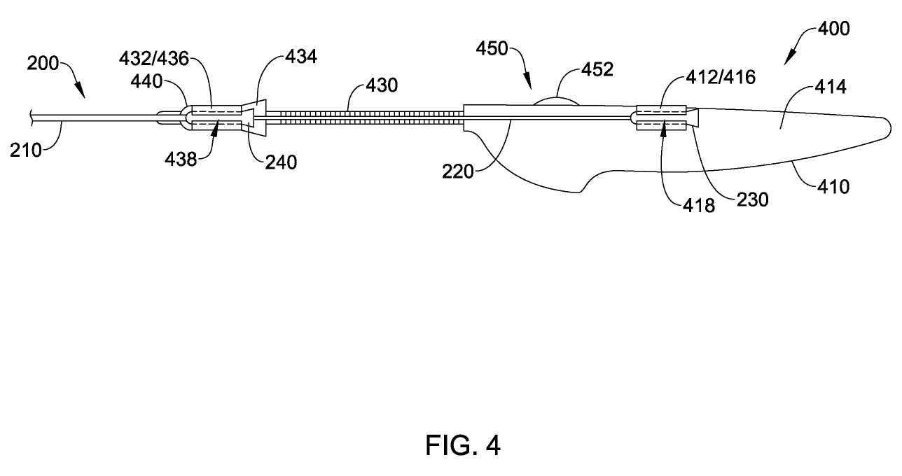

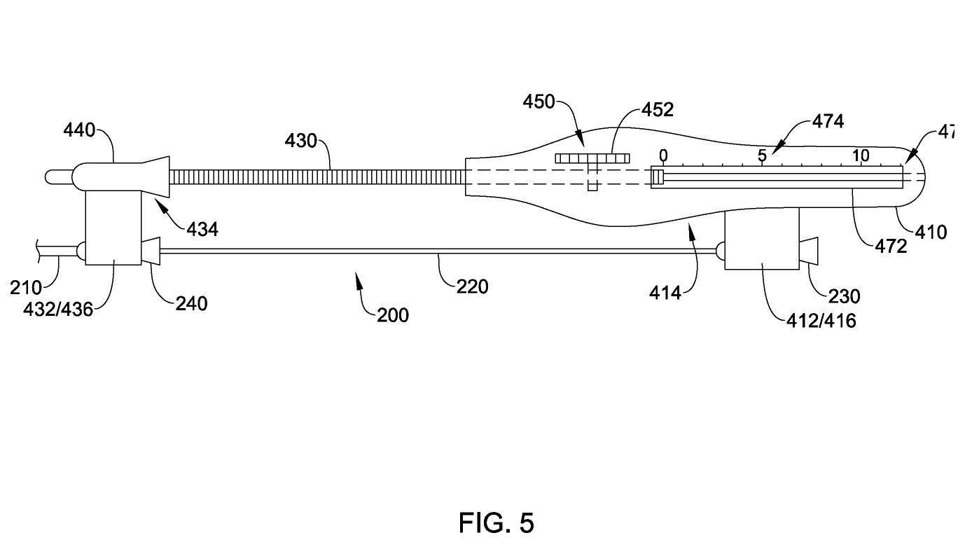

In some embodiments, the stent delivery system 100 may comprise a delivery aid 400 for use with a stent delivery catheter, such as the stent delivery catheter 200 disclosed herein. In some embodiments, the delivery aid 400 may be configured for use with a plurality of different stent delivery catheters. For example, in some embodiments, the delivery aid 400 may be usable with a variety of stent delivery catheters from one or more manufacturers, etc. Other configurations are also contemplated. FIGS. 3-4 are additional views illustrating selected aspects of the delivery aid 400.

In some embodiments, the delivery aid 400 may comprise a handle 410. In some embodiments, the handle 410 may comprise a shell having an interior space disposed therein. In some embodiments, the handle 410 may comprise a first half 411A and a second half 411B, as seen in FIG. 3. During normal use, the first half 411A and the second half 411B may be secured and/or fixed together, such as with fasteners or adhesive. Other configurations are also contemplated.

In some embodiments, the delivery aid 400 may comprise a first delivery system receiver 412 attachable to and/or attached to the handle 410 of the delivery aid 400. In some embodiments, the first delivery system receiver 412 may be fixedly attached to the handle 410 of the delivery aid 400. Other configurations are also contemplated.

In some embodiments, the first delivery system receiver 412 may be provided on and/or disposed on an exterior surface 414 of the handle 410. In some embodiments, the first delivery system receiver 412 may include a first receptacle 416. In at least some embodiments, the first receptacle 416 may comprise a first lateral opening 418 (e.g., FIG. 4). The first delivery system receiver 412 and/or the first receptacle 416 may be configured to removably receive and/or releasably couple to a first portion of the stent delivery catheter 200. In some embodiments, the first portion of the stent delivery catheter 200 may be the first handle 230 of the stent delivery catheter 200. In some embodiments, the first delivery system receiver 412 and/or the first receptacle 416 may be configured to removably receive and/or removably secure the first handle 230 of the stent delivery catheter 200 therein. In some embodiments, the first lateral opening 418 may be configured to removably receive the first handle 230 of the stent delivery catheter 200 laterally therein.

In some embodiments, the first delivery system receiver 412 and/or the first receptacle 416 comprise a C-shaped structure configured to receive the first portion of the stent delivery catheter 200 and/or the first handle 230 of the stent delivery catheter 200 therein. In some embodiments, the first delivery system receiver 412 and/or the first receptacle 416 may be configured to snap onto the first handle 230 of the stent delivery catheter 200, and/or the first handle 230 may be configured to snap into the first delivery system receiver 412 and/or the first receptacle 416. In some alternative embodiments, the first handle 230 of the stent delivery catheter 200 may be configured to slide into the first delivery system receiver 412 and/or the first receptacle 416 axially. In some further alternative embodiments, the first delivery system receiver 412 and/or the first receptacle 416 may comprise a clamp configured to close around the first portion of the stent delivery catheter 200 and/or the first handle 230 of the stent delivery catheter 200. In some embodiments, the clamp may be configured to surround the first portion of the stent delivery catheter 200 and/or the first handle 230 of the stent delivery catheter 200. Other configurations are also contemplated.

In some embodiments, the delivery aid 400 may comprise an elongate shaft 430 extending distally from the handle 410. In some embodiments, the elongate shaft 430 may be slidably disposed on and/or over a guide shaft 431. In some embodiments, the guide shaft 431 may be attached and/or secured to the handle 410. In one example, the guide shaft 431 may be attached and/or secured to the handle 410 at a proximal end of the handle 410. Other configurations are also contemplated. The elongate shaft 430 may be configured to slide axially and/or longitudinally along and/or over the guide shaft 431. In some embodiments, the guide shaft 431 may extend completely through the elongate shaft 430. In some embodiments, a proximal end of the guide shaft 431 may extend proximally of a proximal end of the elongate shaft 430. In some embodiments, a distal end of the guide shaft 431 may extend distally of a distal end of the elongate shaft 430. Other configurations are also contemplated.

In some embodiments, the delivery aid 400 may comprise a second delivery system receiver 432 attachable to and/or attached to the elongate shaft 430 of the delivery aid 400. In some embodiments, the second delivery system receiver 432 may be fixedly attached to the elongate shaft 430 of the delivery aid 400. Other configurations are also contemplated.

In some embodiments, the second delivery system receiver 432 may be provided on and/or disposed on an exterior surface 434 of the elongate shaft 430. In some embodiments, the second delivery system receiver 432 may include a second receptacle 436. In at least some embodiments, the second receptacle 436 may comprise a second lateral opening 438 (e.g., FIG. 4). The second delivery system receiver 432 and/or the second receptacle 436 may be configured to removably receive and/or releasably couple to a second portion of the stent delivery catheter 200. In some embodiments, the second portion of the stent delivery catheter 200 may be the second handle 240 of the stent delivery catheter 200. In some embodiments, the second delivery system receiver 432 and/or the second receptacle 436 may be configured to removably receive and/or removably secure the second handle 240 of the stent delivery catheter 200 therein. In some embodiments, the second lateral opening 438 may be configured to removably receive the second handle 240 of the stent delivery catheter 200 laterally therein.

In some embodiments, the second delivery system receiver 432 and/or the second receptacle 436 comprise a C-shaped structure configured to receive the second portion of the stent delivery catheter 200 and/or the second handle 240 of the stent delivery catheter 200 therein. In some embodiments, the second delivery system receiver 432 and/or the second receptacle 436 may be configured to snap onto the second handle 240 of the stent delivery catheter 200, and/or the second handle 240 may be configured to snap into the second delivery system receiver 432 and/or the second receptacle 436. In some alternative embodiments, the second handle 240 of the stent delivery catheter 200 may be configured to slide into the second delivery system receiver 432 and/or the second receptacle 436 axially. In some further alternative embodiments, the second delivery system receiver 432 and/or the second receptacle 436 may comprise a clamp configured to close around the second portion of the stent delivery catheter 200 and/or the second handle 240 of the stent delivery catheter 200. In some embodiments, the clamp may be configured to surround the second portion of the stent delivery catheter 200 and/or the second handle 240 of the stent delivery catheter 200. Other configurations are also contemplated.

In some embodiments, the first delivery system receiver 412 and/or the first receptacle 416 and the second delivery system receiver 432 and/or the second receptacle 436 may be configured to position the stent delivery catheter 200 entirely outside of and/or exterior to the handle 410 of the delivery aid 400. In some embodiments, the first delivery system receiver 412 and/or the first receptacle 416 and the second delivery system receiver 432 and/or the second receptacle 436 may be configured to position the stent delivery catheter 200 entirely outside of and/or exterior to the elongate shaft 430 of the delivery aid 400.

In some embodiments, the delivery aid 400 may comprise an actuation mechanism 450 disposed at least partially within the handle 410. In some embodiments, the actuation mechanism 450 may comprise a thumb wheel 452 operatively engaged with the elongate shaft 430. In some embodiments, the elongate shaft 430 may comprise a linear gear 454 disposed along a length of the elongate shaft 430. In some embodiments, the thumb wheel 452 may comprise a circular gear 456 (e.g., FIG. 3) engaged with and/or configured to engage with the linear gear 454. In some embodiments, the actuation mechanism 450 may collectively comprise the thumb wheel 452, the linear gear 454, and/or the circular gear 456.

The actuation mechanism 450 and/or the thumb wheel 452 may be configured to axially translate the second portion of the stent delivery catheter 200 relative to the first portion of the stent delivery catheter 200. In some embodiments, the actuation mechanism 450 and/or the thumb wheel 452 may be configured to axially translate the second portion of the stent delivery catheter 200 relative to the first portion of the stent delivery catheter 200 with the stent delivery catheter 200 disposed entirely outside of and/or exterior to the handle 410 of the delivery aid 400.

In some embodiments, the actuation mechanism 450 and/or the thumb wheel 452 may be configured to axially translate the second delivery system receiver 432 and/or the second receptacle 436 relative to the first delivery system receiver 412 and/or the first receptacle 416. In some embodiments, the actuation mechanism 450 and/or the thumb wheel 452 may be configured to axially translate the second delivery system receiver 432 and/or the second receptacle 436 relative to the first delivery system receiver 412 and/or the first receptacle 416 to deploy the stent 300 from the stent delivery catheter 200. In some embodiments, actuation of the actuation mechanism 450 and/or the thumb wheel 452 may be configured to axially translate the second delivery system receiver 432 and/or the second receptacle 436 relative to the first delivery system receiver 412 and/or the first receptacle 416 to deploy the stent 300 from the stent delivery catheter 200. In some embodiments, actuation of the actuation mechanism 450 and/or the thumb wheel 452 may be configured to axially translate the outer tubular member 210 of the stent delivery catheter 200 relative to the inner tubular member 220 of the stent delivery catheter 200 to deploy the stent 300 from the stent delivery catheter 200. In some embodiments, actuation of the actuation mechanism 450 and/or the thumb wheel 452 may be configured to axially translate the second handle 240 of the stent delivery catheter 200 relative to the first handle 230 of the stent delivery catheter 200 to deploy the stent 300 from the stent delivery catheter 200.

In some embodiments, the actuation mechanism 450 may be configured for measured deployment of the stent 300 from the stent delivery catheter 200. In some embodiments, the actuation mechanism 450 may provide precisely measured and/or precisely controlled deployment of the stent 300 from the stent delivery catheter 200. In some embodiments, the delivery aid 400 and/or the actuation mechanism 450 may provide improved accuracy and control when deploying the stent 300 from the stent delivery catheter 200.

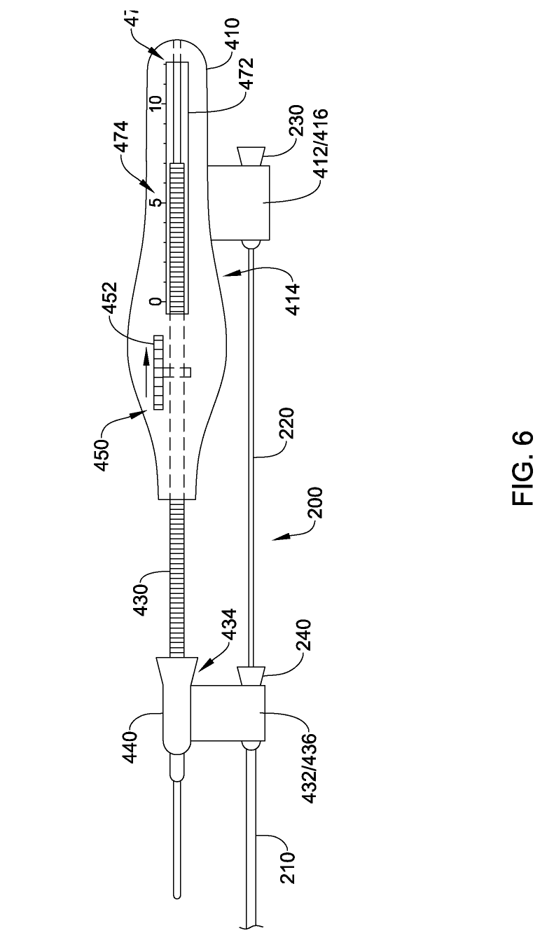

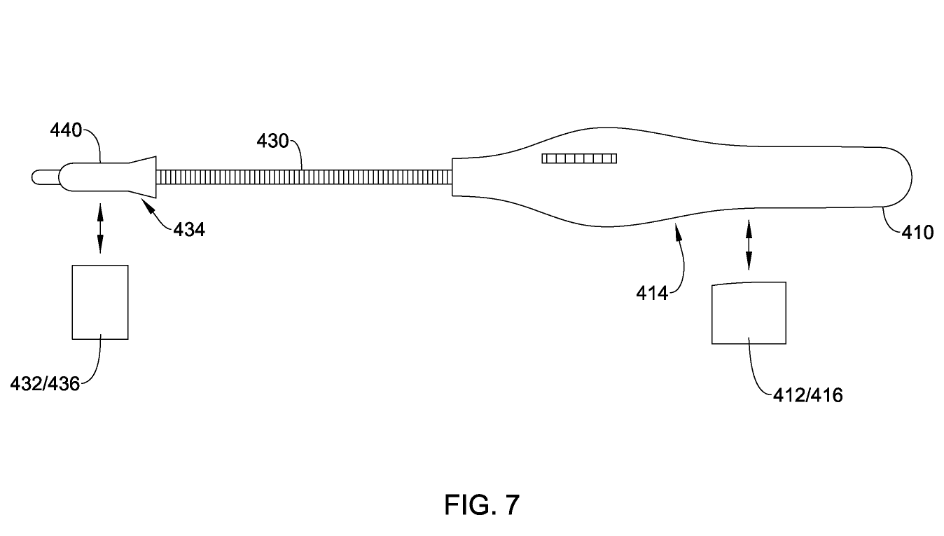

In some embodiments, the handle 410 may comprise a visual aid 470, as seen in FIGS. 5-6. In some embodiments, the visual aid 470 may be configured to show how much of the stent 300 disposed within the stent delivery catheter 200 has been deployed. In some embodiments, the visual aid 470 may comprise a window 472 formed in and/or extending through a wall of the handle 410. In some embodiments, the visual aid 470 may comprise a linear scale 474 disposed alongside the window 472. In some embodiments, the linear scale 474 may be printed on the exterior surface 414 of the handle 410. In some embodiments, the linear scale 474 may be formed in the wall of the handle 410. For example, the linear scale 474 may be molded into the wall of the handle 410. Other configurations are also contemplated. In some embodiments, the elongate shaft 430 may be visible through the window 472. Accordingly, as the elongate shaft 430 axially translates relative to the handle 410, an amount of the elongate shaft 430 visible through the window 472 may change, as seen in FIGS. 5-6. The user may use the visual aid 470 to determine and/or track how much of the stent 300 disposed within the stent delivery catheter 200 has been deployed. For example, if the numbers shown on the linear scale 474 in FIGS. 5-6 are considered to represent millimeters, FIG. 5 shows that none (e.g., zero millimeters) of the stent 300 has been deployed (such as shown in FIG. 1), while FIG. 6 shows that approximately 7 millimeters of the stent 300 has been deployed (e.g., 7 millimeters of the stent 300 is not covered by the outer tubular member 210). The linear scale 474 shown on the handle 410 in FIGS. 5-6 is merely exemplary, and other values, units, etc. are also contemplated. Additionally, the visual aid 470 may take other forms including, but not limited to, a digital display, a dial, etc.

In some embodiments, the first delivery system receiver 412 and/or the first receptacle 416 may be detachable from the handle 410, as seen in FIG. 7. In some embodiments, the second delivery system receiver 432 and/or the second receptacle 436 may be detachable from the elongate shaft 430, as seen in FIG. 7. In some embodiments, the delivery aid 400 may comprise and/or may be provided with a plurality of first delivery system receivers and a plurality of second delivery system receivers, wherein each first delivery system receiver is compatible with and/or attachable to the handle 410 and each second delivery system receiver is compatible with and/or attachable to the elongate shaft 430. The plurality of first delivery system receivers and the plurality of second delivery system receivers may be configured for use with varying configurations and/or sizes of stent delivery catheters such that the delivery aid 400 may be used with a variety of stent delivery catheters from one or more manufacturers, etc. As such, the user may choose the first delivery system receiver and the second delivery system receiver configured for use with their chosen stent delivery catheter.

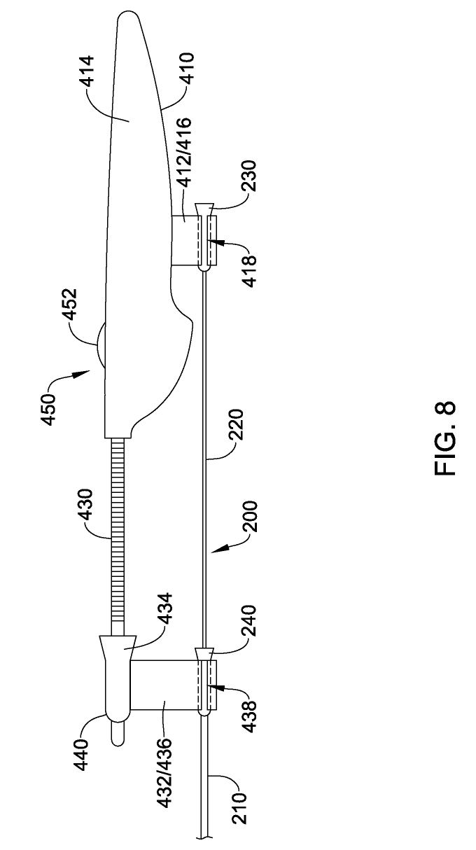

In some embodiments, the delivery aid 400 may be configured for single-handed use. In some embodiments, the handle 410 of the delivery aid 400 may be configured for ambidextrous operation of the actuation mechanism 450. In some embodiments, the first delivery system receiver 412 and/or the first receptacle 416 may be disposed on and/or along a bottom side of the handle 410 such that the handle 410 may be grasped with either a left hand or a right hand without negatively impacting operation of the actuation mechanism 450, as seen in FIG. 8. Similarly, the second delivery system receiver 432 and/or the second receptacle 436 may be disposed on and/or along a bottom side of the elongate shaft 430. Depending on the shape and/or form of the handle 410, the first delivery system receiver 412 and/or the first receptacle 416, and the second delivery system receiver 432 and/or the second receptacle 436, may have a different form factor than other embodiments in order to prevent interference between the handle 410 and the stent delivery catheter 200.

Returning briefly to FIGS. 1-2, in some embodiments, the elongate shaft 430 may comprise a distal handle 440 configured to be gripped by a second hand of a user of the delivery aid 400. For example, if the user’s right hand is on the handle 410, the user’s left hand may be placed on the distal handle 440. Alternatively, if the user’s left hand is on the handle 410, the user’s right hand may be placed on the distal handle 440.

The distal handle 440 may be fixedly attached to the elongate shaft 430 proximate a distal end of the elongate shaft 430. In some embodiments, the distal handle 440 may be monolithically formed with the elongate shaft 430. In some embodiments, the second delivery system receiver 432 and/or the second receptacle 436 may be provided on and/or disposed on an exterior surface of the distal handle 440. In some embodiments, the exterior surface 434 of the elongate shaft 430 may be the exterior surface of the distal handle 440. In some embodiments, the second delivery system receiver 432 and/or the second receptacle 436 may be attached directly to the elongate shaft 430. In some embodiments, the second delivery system receiver 432 and/or the second receptacle 436 may be attached directly to the distal handle 440.

In some embodiments, the distal handle 440 and the handle 410 may be configured for “rapid deployment” of the stent 300 via manually manipulating the elongate shaft 430 relative to the handle 410 of the delivery aid 400 using two-handed operation and/or without using the thumb wheel 452. In some embodiments, the distal handle 440 may be configured to override the actuation mechanism 450 and/or the thumb wheel 452 (e.g., the second portion of the stent delivery catheter 200 may be axially translated relative to the first portion of the stent delivery catheter 200 by moving the distal handle 440 relative to the handle 410 instead of actuating the thumb wheel 452). For example, with two-handed operation via the distal handle 440, the actuation mechanism 450 and/or the thumb wheel 452 may be slaved to the distal handle 440 (e.g., the distal handle 440 controls and/or causes operation of the actuation mechanism 450 and/or axial translation of the second portion of the stent delivery catheter 200 relative to the first portion of the stent delivery catheter 200). The linear gear 454 and the circular gear 456 may permit manual movement of the elongate shaft 430 relative to the handle 410 of the delivery aid 400 without using the thumb wheel 452, whereas a helical gear and/or a screw-type mechanism may resist such movement unless rotated directly using the thumb wheel 452.

The materials that can be used for the various components of the stent delivery system and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the delivery aid, the stent delivery catheter, the stent, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP).

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A delivery aid for use with a stent delivery catheter, comprising: a handle; an elongate shaft extending distally from the handle; a first delivery system receiver attached to the handle and configured to releasably couple to a first portion of the stent delivery catheter; a second delivery system receiver attached to the elongate shaft distal of the handle and configured to releasably couple to a second portion of the stent delivery catheter; and an actuation mechanism disposed at least partially within the handle, wherein the actuation mechanism is configured to axially translate the second portion of the stent delivery catheter relative to the first portion of the stent delivery catheter with the stent delivery catheter disposed entirely outside of the handle.

2. The delivery aid of claim 1, wherein the first delivery system receiver is provided on an exterior surface of the handle.

3. The delivery aid of claim 1, wherein the second delivery system receiver is provided on an exterior surface of the elongate shaft.

4. The delivery aid of claim 1, wherein the first delivery system receiver comprises a first lateral opening configured to receive the first portion of the stent delivery catheter laterally therein.

5. The delivery aid of claim 4, wherein the first portion of the stent delivery catheter is a first handle of the stent delivery catheter attached to a proximal end of an inner tubular member of the stent delivery catheter, wherein the first delivery system receiver includes a first receptacle configured to removably secure the first handle of the stent delivery catheter therein.

6. The delivery aid of claim 1, wherein the second delivery system receiver comprises a second lateral opening configured to receive the second portion of the stent delivery catheter laterally therein.

7. The delivery aid of claim 6, wherein the second portion of the stent delivery catheter is a second handle of the stent delivery catheter attached to a proximal end of an outer tubular member of the stent delivery catheter, wherein the second delivery system receiver includes a second receptacle configured to removably secure the second handle of the stent delivery catheter therein.

8. The delivery aid of claim 1, wherein the actuation mechanism includes a thumb wheel operatively engaged with the elongate shaft.

9. The delivery aid of claim 8, wherein rotation of the thumb wheel is configured to axially translate the second delivery system receiver relative to the first delivery system receiver.

10. The delivery aid of claim 9, wherein the elongate shaft comprises a linear gear and the thumb wheel comprises a circular gear engaged with the linear gear.

11. The delivery aid of claim 1, wherein the handle comprises a visual aid configured to show how much of a stent disposed within the stent delivery catheter has been deployed.

12. The delivery aid of claim 11, wherein the visual aid comprises a window formed in a wall of the handle and a linear scale disposed alongside the window.

13. The delivery aid of claim 1, wherein the first delivery system receiver is detachable from the handle and the second delivery system receiver is detachable from the elongate shaft.

14. A delivery aid for use with a stent delivery catheter, comprising a handle; an actuation mechanism disposed at least partially within the handle; a first receptacle configured to removably receive a first handle of the stent delivery catheter therein; and a second receptacle configured to removably receive a second handle of the stent delivery catheter therein; wherein the actuation mechanism is configured to axially translate the second receptacle relative to the first receptacle to deploy a stent from the stent delivery catheter.

15. The delivery aid of claim 14, wherein the first receptacle comprises a first lateral opening configured to receive the first handle of the stent delivery catheter laterally therein.

16. The delivery aid of claim 15, wherein the second receptacle comprises a second lateral opening configured to receive the second handle of the stent delivery catheter laterally therein.

17. A stent delivery system, comprising: a stent delivery catheter comprising: an outer tubular member; an inner tubular member slidably disposed within the outer tubular member; a first handle secured to a proximal end of the inner tubular member; and a second handle secured to a proximal end of the outer tubular member; a stent radially constrained between the inner tubular member and the outer tubular member; and a delivery aid comprising: a handle; an actuation mechanism disposed at least partially within the handle; a first receptacle configured to removably receive the first handle of the stent delivery catheter therein; and a second receptacle configured to removably receive the second handle of the stent delivery catheter therein; wherein actuation of the actuation mechanism is configured to axially translate the outer tubular member relative to the inner tubular member to deploy the stent.

18. The stent delivery system of claim 17, wherein the first receptacle and the second receptacle are configured to position the stent delivery catheter entirely outside of the handle of the delivery aid.

19. The stent delivery system of claim 17, wherein the actuation mechanism is configured for measured deployment of the stent from the stent delivery catheter.

20. The stent delivery system of claim 17, wherein the handle is configured for ambidextrous operation of the actuation mechanism.
